(19) United States
(12) Patent Application Publication
(10) Pub. No.: US 2026/0207672 A1
(43) Pub. Date: Jul. 23, 2026

(54) STEM CELL REPROGRAMMING WITH GENE-ENHANCED STEM CELLS

(71) Applicants: Vincent C. Giampapa, Montclair, NJ (US); Elizabeth L. Parrish, Bainbridge Island, WA (US); Victor Urzola, San Rafae (CR)

(72) Inventors: Vincent C. Giampapa, Montclair, NJ (US); Elizabeth L. Parrish, Bainbridge Island, WA (US); Victor Urzola, San Rafae (CR)

(21) Appl. No.: 19/387,418

(22) Filed: Nov. 12, 2025

Related U.S. Application Data
(63) Continuation of application No. PCT/US2025/012415, filed on Jan. 21, 2025.
Application No. PCT/US2025/012415 is a continuation of application No. 18/982,680, filed on Dec. 16, 2024.
(60) Provisional application No. 63/690,466, filed on Sep. 4, 2024.

(51) Int. Cl. A61K 35/28; C07K 14/47; C12N 5/074; C12N 5/0775; C12N 9/02; C12N 9/12; C12N 9/24; C12N 15/86

(52) U.S. Cl. CPC A61K 35/28; C07K 14/4703; C12N 5/0662; C12N 5/0696; C12N 9/0071; C12N 9/1276; C12N 9/2402; C12N 15/86; C12N 2510/00; C12N 2710/16143; C12N 2750/14143; C12Y 114/11; C12Y 207/07049; C12Y 302/01031

(57) ABSTRACT

Using adults stem cells (ASC) including hematopoietic cell (HSC), endothelial progenitor cells and mesenchymal (MSC) stem cells; mobilizing ASC cells into the vascular system using a collection system of one of: (i) an apheresis. (ii) bone marrow; or (iii) direct collection of ASC from the peripheral blood; providing the ASC with a cryopreservation conservation container of about minus 80 degrees centigrade collecting the ASC for defrosting and placed in a sterile container; reprogramming stem cells (RSC) including at least one of (i) RSC by methylation patterns of DNA such as JMJD3 and HDAC; (ii) RSC by acetylation patterns of DNA such as HDAC; (iii) epigenetic programming with a protein of interest (ncRNA); using vectors of AA V or CMV to transfer to specific genes to provide proteins to enlarge physical conditions to lost during the aging process.

### CROSS-REFERENCE TO RELATED APPLICATIONS

This application is a continuation under 35 U.S.C. § 111 of International Application No. PCT/US2025/012415, filed 21 Jan. 2025, which claims priority to U.S. patent application Ser. No. 18/982,680, filed 16 Dec. 2024, which claims priority to U.S. Provisional Ser. No. 63/690,466 filed September 4 2024. Each of the foregoing applications is incorporated herein by reference in its entirety.

### TECHNICAL FIELD

The present disclosure generally relates to methodologies for the reprogramming of stem cells. More specifically this disclosure relates to the therapeutic utilization of these methodologies.

Stem cells have shown great promise in regenerative medicine for multiple different disease processes caused by the general effects of the cellular aging process. At the same time genetic therapies alone have also been shown to be significantly important in treatments used for specific inherited disease states as well as cancer.

The use of therapies, including the product of adult stem cells (ASC), has also been shown to have regenerative effects on both cell function and certain diseases related to the aging process. (ASC) may use hematopoietic cell (HSC), endothelial progenitor cells and mesenchymal (MSC) stem cells. Such cells can be differentiated into a variety of cells including osteoblasts (bone cells), chondrocytes (cartilage cells), myocytes (muscle cells) and adipocytes (fat or marrow cells), as well as all immune cell types.

This invention describes the concept of enhancing stem cell function with a specific manufactured human gene type which will allow for the regenerative potential of stem cells to be greatly enhanced by combining both gene and stem cell therapies together. One may mobilize ASC cells into the vascular system using an infusion collection system as set forth below. The combination of these nascent technologies creates a synergistic effect for regenerative cellular therapy far beyond their effectiveness alone. This also entails the collection of ASC with a cryopreservation conservation container of about minus 80 degrees Centigrade (minus 112 degrees F).

As a further step reprogrammed stem cells (RSC) may include the following of RSC by methylation patterns of DNA; RSC by acetylation patterns of DNA; and epigenetic programming including RNA splicing with a protein of interest (ncRNA).

The method uses vectors of at least AA V or CMV to transfer specific genes to provide proteins necessary to regenerate physical condition that otherwise are lost during the aging process, using particular vectors as set forth in the Summary of the Invention. The programming of ASC can be used by reinfusion of (RSC) by using direct injection of RSC along with using gene enhancement of the RSC.

In addition, the use of exosome-derived macrovesicles has been successfully used by the present inventors Vincent Giampapa and Linda Crouse, Increasing Telomere Length in a Cell of U.S. Pat. No.10,098,922 (2018). Also, inventors Steven J. Greco and Vincent Giampapa, Cell Free Compositions for Cellular Restoration and Methods of Making and Using Same of U.S. Pat. No. 10,772,911 (2020). And inventor Vincent Giampapa, Compositions for Cellular Restoration and Methods of Making and Using Same of U.S. Pat. No. 11,219,643 (2022) and further inventors Steven J. Greco and Vincent Giampapa, Reprogramming of Aged Adult Stem Cells of U.S. Pat. No. 11,286,463 (2022).

In other pertinent part is U.S. 2014/0010801 A1 of Niedernhofer, et al regarding Composition and Methods or Restoring or Rejuvenating Stem Cells. Also, Inventors are Douglas Coyle and Michael Fessel, Compositions and Methods for Providing Active Telomerase to Cells in Vivo, Patent Application No. 2016/0324986 (2016); Inventor Ohio State Innovation Foundation, RNA Ligand-Displaying Exosomes for Specific Deliver, Patent Application WO 2017/176894 (2017); and is also U.S. 2022/0251603 to Parrish, et al regarding System and Methods for Gene Therapy via Genetically Modified Viral Vectors. And yet further U.S. 2022/0325258 of Kogut, et al regarding Methods for Cell and Tissue Rejuvenation; and U.S. 2022/0136011 of Kalluri for Telomerase-Containing Exosomes for Treatment of Age-Related Organ Dysfunction. Also, China U.S. Patent No. 112695049 of Minglu, et al., for Fusion Gene and Plasmid for Mesenchymal Stem Cells.

### SUMMARY OF THE INVENTION

An adult stem cell therapy includes using adults stem cells (ASC) including hematopoietic cells (HSC), endothelial progenitor cells and mesenchymal (MSC) stem cells; mobilizing ASC cells into the vascular system using a collection system of one approaches: (i) an apheresis; (ii) bone marrow; or (iii) direct collection of ASC from the peripheral blood; also providing the ASC with a cryopreservation conservation container of about minus 80 degrees centigrade (about minus 112 degrees F); collecting the ASC for defrosting and placed in a sterile container; reprograming stem cells (RSC) including at least one of the following: (i) RSC by methylation patterns of DNA such as JMJD3 proteins and HDAC; or (ii) RSC by acetylation patterns of DNA such as HDAC; using vectors of at least AAV or CMV to transfer to specific genes to provide proteins necessary to regenerate physical conditions that otherwise are lost during the aging process, including at least one of: (i) gene enhanced MSN cells with follistatin (ii) hTERT with enhanced HSC cells; or (iii) klotho cells with enhanced MSN with or without ultrasound therapy; and (iv) gene enhanced ASC with PGC1-alpha, programming of stem cells as set forth: (i) reinfused using reprogrammed stem cells (RSC); (ii) using direct injection of RSC; or (iii) using gene enhanced RSC, whereas an IV peripheral line infused into the vein of the person treated with ASC for over a period of about 30 minutes; or whereas the gene enhanced cell (RSC) is directly injected into a specific issue type.

The invention also describes the use of specific human genes, including klotho, follistatin, hTERT, PGC1-alpha, and others not listed here that can be used with exosomes alone forming an exosome-gene complex, or with the combination of stem cell-gene enhancement in combination with exosome-gene complexes.

The focus of this invention is to help restore enhanced cellular function and the physical functional loss occurring in humans due to the general aging process with genetically enhanced stem cells, with or without exosome-gene complexes.

The functional losses in humans over time which are of main concern here include, sarcopenia and frailty, cognitive impairment, central brain nuclei regulatory mechanisms, dementia, immunosenescence, as well as the loss of cellular and general body energy production. All these effects of aging decrease quality of life and health span as well as longevity. They have been well documented in many recent peer reviewed publications. The cellular and physical loss of functions cause a great amount in global healthcare costs in the aging population. This technology will be a large benefit in decreasing the long-term care of the aging population as well as helping them to maintain their jobs and independent function longer.

The aging process causes major functional alterations at the level of our DNA in our stem cells, and somatic cells, turning off key genes that are active when we are young and activating key genes that cause diseases of aging as we grow old. This process occurs in both our body-somatic cells and our stem cells. This results in two major problems, loss of stem cell and somatic cell function and quality and loss of stem cell numbers.

The process to restore the negative impact of the aging process is related to two main conditions. Firstly, the loss of stem cell numbers which can be restored. This loss is due mainly to telomerase shortening, genomic instability of DNA damage and the loss of hTert activity known as telomerase transcription factor which is a catalytic subunit of the enzyme telomerase. It together with the telomerase RNA component (TERC) which is the most important unit of the telomerase complex.

Secondly, the loss of both stem cell and somatic cell functional quality due to changes in methylation and acetylation patterns in our DNA over time in both of these cells. These changes in methylation and acetylation occur and alter gene activity due to changes in DNA transcription and optimal youthful protein production in every cell of our body including both stem cells and somatic cells. The changes in proteins at the level of DNA are a major cause of age-related disease and functional loss as time goes by.

This patent application describes a multi-step process that can restore both two pivotal events within our genome and delay and or reverse these changes that cause most of the age-related physical conditions humans suffer from as they age. The results of restoring these processes create an increase in physical functional quality and an increase in health-span and longevity.

### BRIEF DESCRIPTION OF THE DRAWINGS

FIG. 1 is a schematic view of an animal cell (eukaryotic) including the nucleuses, mitochondrion, ribosomes, secretory vesicles, lysosome and cytoplasm.

FIG. 2 is a schematic view of the basic structure of an MSC cell including exosomes depicting the surface and core proteins along with certain immune and growth factors.

FIG. 3 is a representative structure and composition of an exosome and related components thereof.

FIG. 4 Is a schematic view of a cell of the nucleus, chromosome, histones, genes and DNA structure.

FIGS. 5, 6 and 7 are views of DNA, nucleosomes, histones and chromatin (closed and opened).

FIG. 8 is a schematic view showing a DNA to mRNA transcription and into protein synthesis.

FIG. 9 is a schematic view of the use of viral DNA and vector binding to cell membrane and the process thereafter set forth below.

FIG. 10 is a representative view of a direct delivery of transgenes into a patient.

FIG. 11 is a schematic view of the aging clocks of the human body.

### DETAILED DESCRIPTION OF THE INVENTION

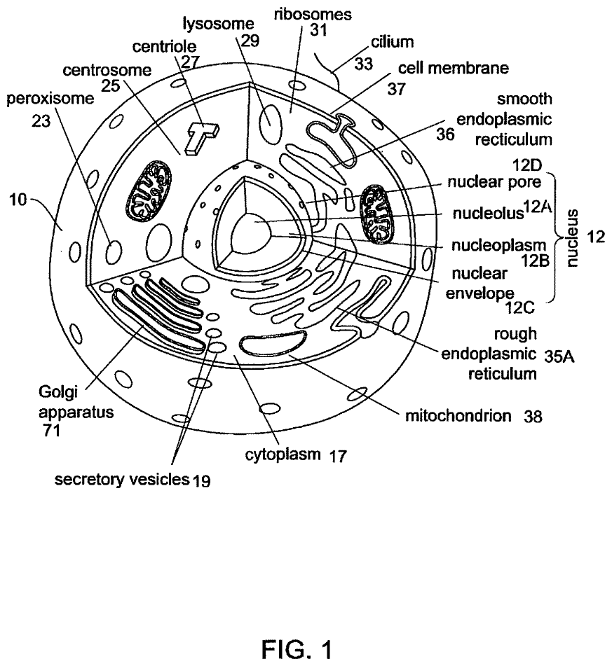

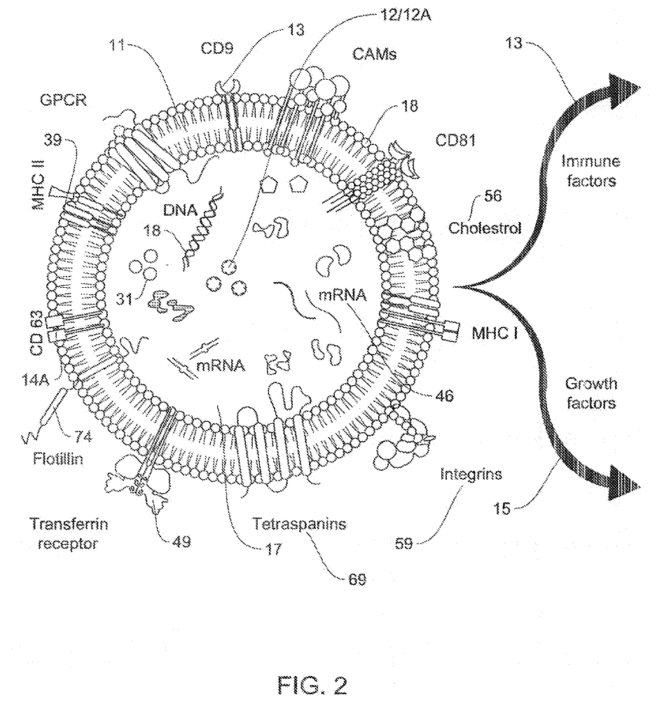

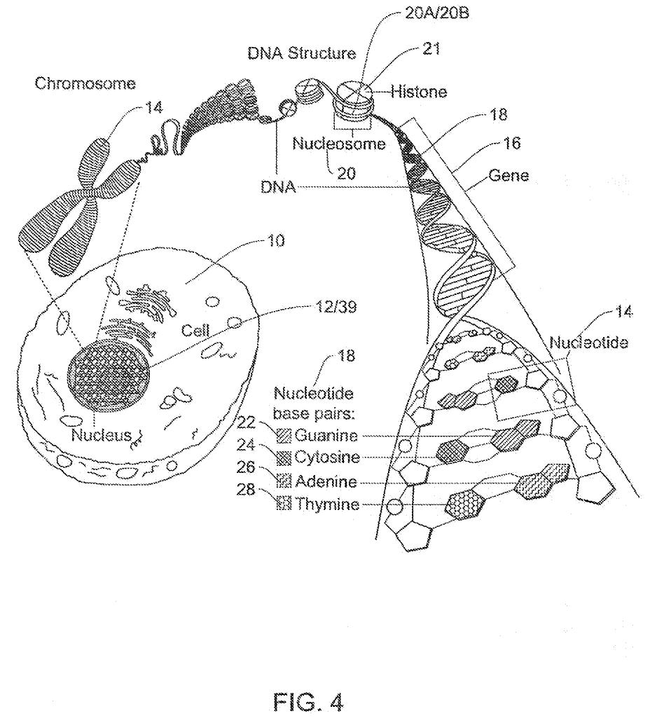

FIG. 1 shows a eukaryotic cell 10 which includes a nucleus 12, a nucleolus 12A, a nucleoplasm 128, the nuclear envelope 12C, nuclear pores 12D, cytoplasm 17, secretory vesicles 19, Golgi apparatus 71, peroxisome (PGC-1 alpha) 23, centrosome 25, centriole 27, lysosome 29, ribosomes 31, cilium 33, smooth endoplasmic reticulum 35, rough endoplasmic reticulum (ER) 35A and cell membrane 37 and mitochondrion 38. The Golgi apparatus 71 is a cell organelle processing proteins and lipids for use in the cell, peroxisome 23 carries oxidative reactions using molecular oxygen. The centrosome 25 maintains equal distribution of chromosomes 14 (see FIG. 4) in daughter cells. Centriole 27 organizes microtubes that serve in the cells'skeletal system. The lysosome 29 functions as the digestive system of the cell. Ribosomes 31 functions as a cellular machinery protein synthesis, thus translating the small RNA 46 (see FIG. 2) of the genetic code. Cilium 33 which functions as a cellular antenna primarily as a sensory item to receive and interpret signals to the environment. The ER 35/35A is covered with ribosomes 31 and synthesized into proteins for the cell membrane 37 and cell interior 12C (see FIG. 1).

In FIG. 2 there is provided a view of the basic structure of the MSC cell 38 which includes a therapeutic gene 11, the nucleus 12, a immune system 13, cytoplasm 17, DNA 18, chromatin 20C, ribosomes 31, stem cells 36, mitochondrion 38, exosomes 39, RNA 46, cholesterol 56, integrin 59, tetraspanins 69 and follistatin 7 4.

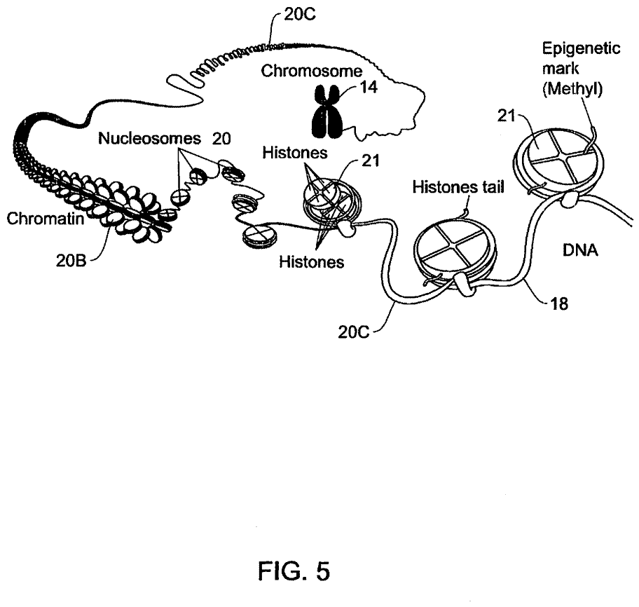

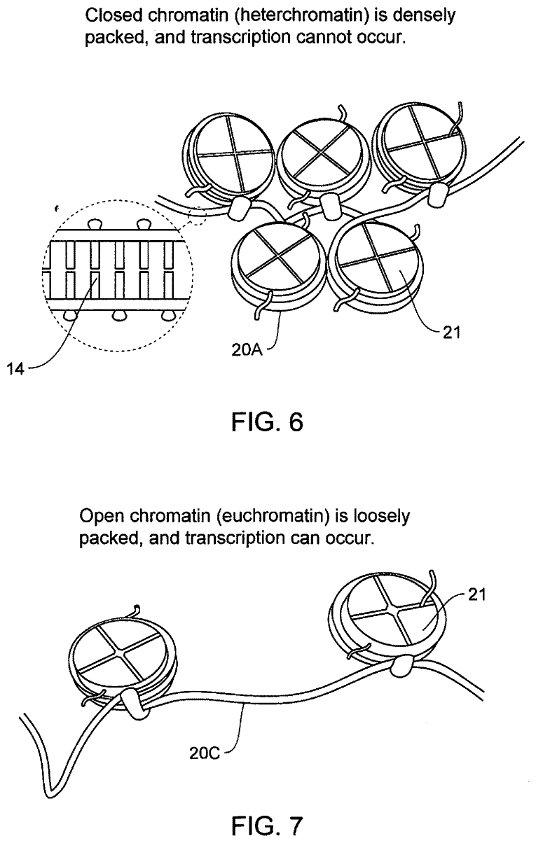

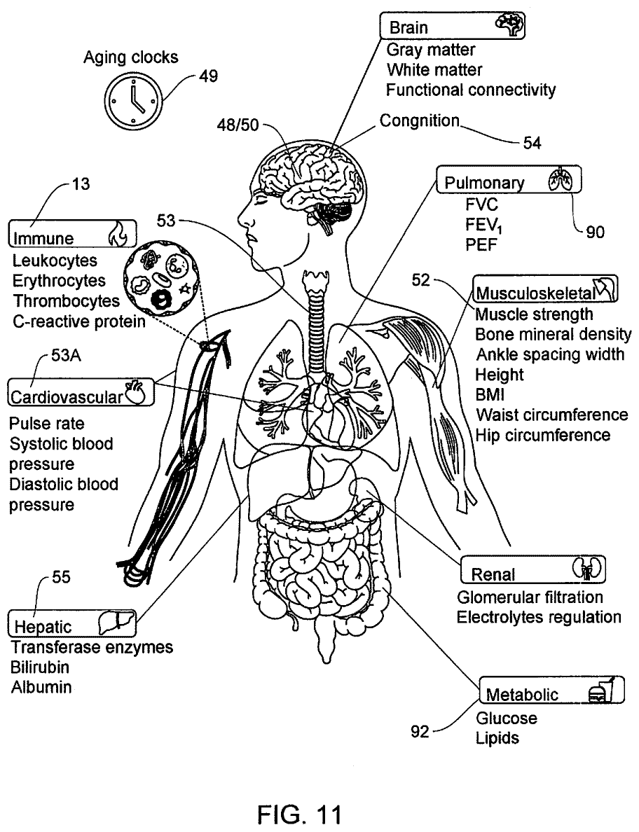

Moving outward from cell 10 in FIGS. 1 and 2 are the DNA structure 18 of FIGS. 4 and 5 which includes a nucleotide 14. Therein is showing a sequence of nucleotide base pairs 18, namely, guanine 22, cytosine 24, adenine 26 and thymine 28. Also showing in FIG. 4 is a klotho gene 41. And also shown are genes 16 which provide triplets of DNA base pairs 18. Also, in FIGS. 4 and 5 is a nucleosome 20 each having a segment of DNA 18 that is around eight (8) histone proteins 21 (methyl) that resemble a spool 20A (see FIG. 4 to 6). This represents a subunit of chromatin 208, the nucleus is compacted into the nucleoplasm 128 and the chromatin 208. These are folded and compacted into each chromosome 14 (see FIGS. 4 and 5). The above of FIG. 6 as the histones packed) 20A open chromatin 20C, nucleoplasm 20 and chromatin 208 (see FIGS. 4, 6 and 7). Histone methylation, as a mechanism for modifying chromatin 20C (see FIG. 7) is associated with stimulation of neural pathways known to be important for formation of long-term memories 54 (see FIG. 11) and learning. Histone methylation 92 is crucial for almost all phases of animal development.

In the present stem therapy process, there are methylation patterns of DNA used for effect by a protein of JMJD3. In JMJD3 an important epigenetic associated with transcriptional silencing is provided, JMJD3 has been studied extensively in immune diseases, cancer, and tumor development. Studies have illustrated that JMJD3 plays a major role in cell fate determination of pluripotent and multipotent stem cells (MSCs). JMJD3 has been found to enhance self-renewal ability and reduce the differentiation capacity of ESCs and MSCs.

In addition, stem cells are essential in production by HDAC or both methylation and acetylation restoration patterns together. There in addition, HDAC activity is fundamental to cellular health. This including histones 21/92 are the most eminent DNA-interacting proteins. As the primary protein constituent of chromatin 20B/20C, they form complexes with DNA to compact our large genome for efficient nuclear organization. Histones support critical cellular processes, such as transcription, DNA replicator, and DNA repair. See FIGS. 5-7.

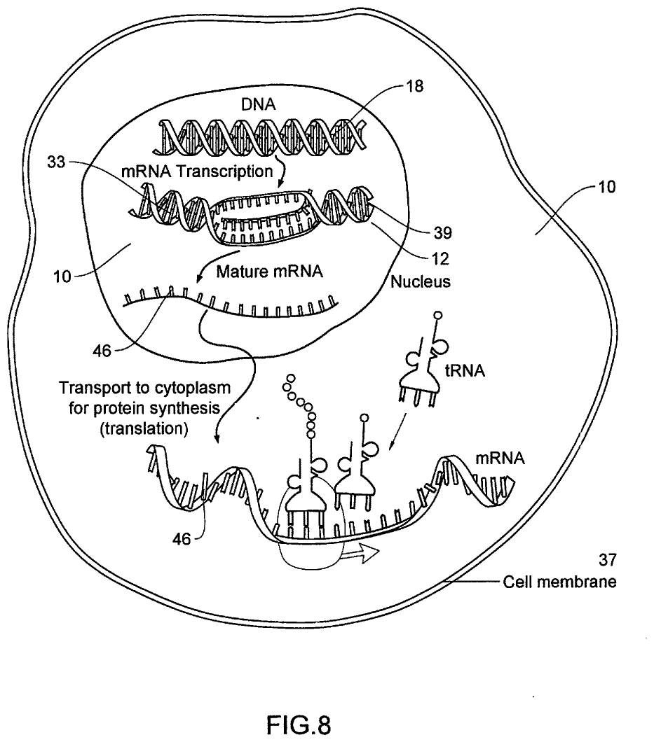

Shown in FIG. 8 is a view of DNA 18, mRNA 46 which is transcribed into protein synthesis. See also FIG. 7.

The present invention includes Step 1 in which ASC includes hematopoietic cells (HSC), endothelial progenitors' cells EPC and mesenchymal stem cells MSC. Any number of processes may be used for MSC to mobilize into the vascular system using one or more of the aphesis, bone marrow, or direct collection of MSC into the peripheral blood. Also, expansion techniques may be used within the method of the invention.

Step 1 also provides ASC with a cryopreservation container having a temperature within a range of between 60 to 100 degrees centigrade, but with an optimal temperature 80 degrees centigrade (minus 112 Fahrenheit). This preserves the genetic activity and profile of the ASC.

Therein a further process is provided of defrosting them, this is within the general blood bank industry (GMP). Such cells can be kept indefinitely for future use and with the added steps set forth below.

In Step 2 the previously collected ASC are defrosted and provided within a sterile container. At that point the process of reprogramming may be used for the cells. There with there exist a number of possibilities, one example of which are showing in U.S. Pat. No. 11,219,643 of Giampapa providing the method of obtaining a first cell sample from a first subject; obtaining a second cell sample from a second subject; culturing the first cell sample in the presence of at least a portion of a culture media of the second cell sample for a time period ranging from about 24 hours to about 6 weeks to produce a restoring composition; and contacting the restoring composition with the second cell sample for a period of time ranging from about 24 hours to about 6 weeks to produce a restored composition.

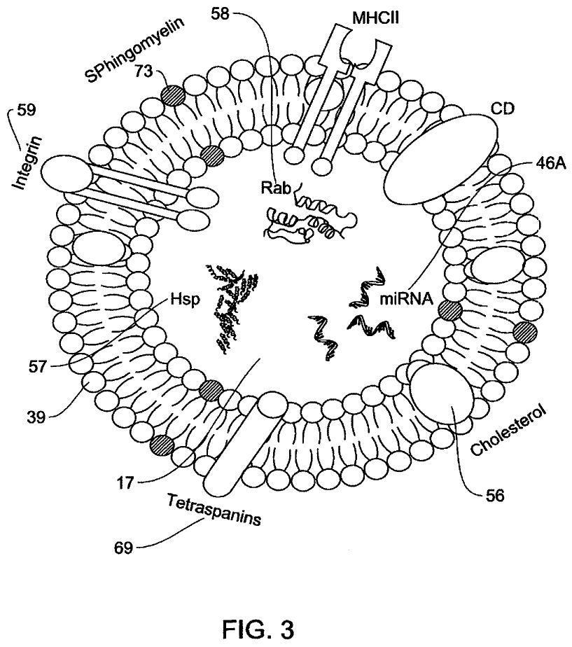

Also, U.S. Pat. No. 10,772,911 (2020) of Greco and Giampapa set forth a pharmaceutical formulation comprising exosomes 39 (see FIG. 2) derived from mobilized stem cells, and one or more pharmaceutically acceptable carriers, adjuvants, or vehicles. A pharmaceutical formulation comprising a secretory vesicle 19 (see FIG. 1) derived from mobilized stem cells, and one or more pharmaceutically acceptable carriers, adjuvants, or vehicles. A pharmaceutical formulation comprising an exosome 39 (see FIG. 3) derived and/or secretory vesicles 19 (see FIG. 1) derived molecule and one or more pharmaceutically acceptable carriers, adjuvants, or vehicles.

In FIG. 3 is shown structure and composition of exosomes 39 at the center is the cells cytoplasm 17. Exosomes 39 include various types of proteins such as HSP 57, Rab 58, lipids or cholesterol 56, Sphingomyelin (SPH) 73, and nucleic acids (miRNA) 46A. SPH surrounds certain nerve cells and is related to the endoplasmic reticulum (ER) 35 (see FIG. 1). The Golgi apparatus 71 can be converted to the SPH 73. Other vesicular components 19 are antigen-presenting cells including integrin 59 and tetraspanins 69 (in FIGS. 2 and 3).

Said patents indicate the use of reprogramming of ASC using the use of trans-well culturing which includes the use of methylation or acetylation patterns in the DNA of stem cells which may accomplish various epigenetic reprogramming processes. This is a safe and natural process of restoring ASC and its somatic cell functions to a more youthful level.

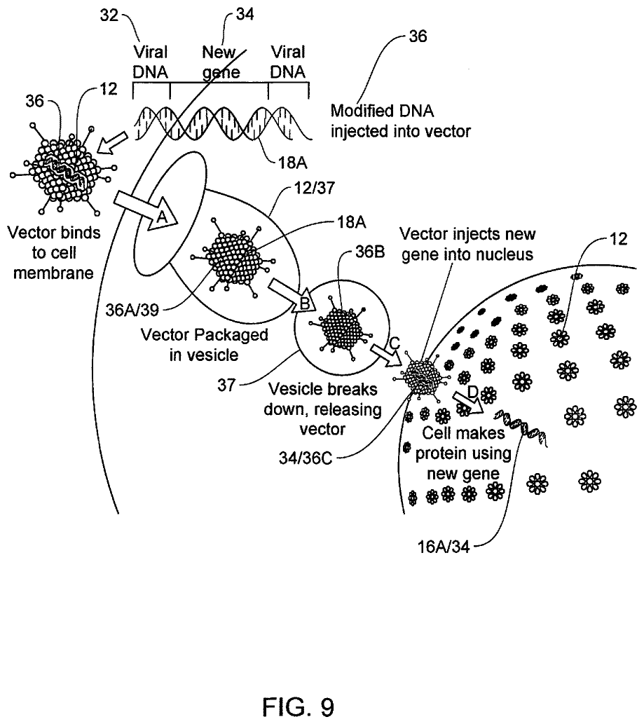

In a further embodiment of Step 3 above, gene vectors 32 (see FIG. 9) can be a viral or plasmid 18A based, as well as other means of, transfection 34 mentioned here. The gene 16 transferred into nucleus 12 does not incorporate into the cellular genetics 10 at any significant levels but remains in the nucleus 12 as an episome 39 or extra piece of DNA 18 (see FIGS. 4 and 9) which will then produce the protein of choice. The protein may leave cell 10 and make its way into the systemic vascular system 53A (see FIG. 11) of the person (see FIG. 11) , or animal, deploying its effects throughout the body. It may also remain in the cell to alter specific functions like increased NAD. It is a co-enzyme in all living cells and is essential for metabolic development and survival of all organisms and/or ATP production or even induce the production of more cellular organelles like mitochondria 38 (see FIGS. 1 and 2).

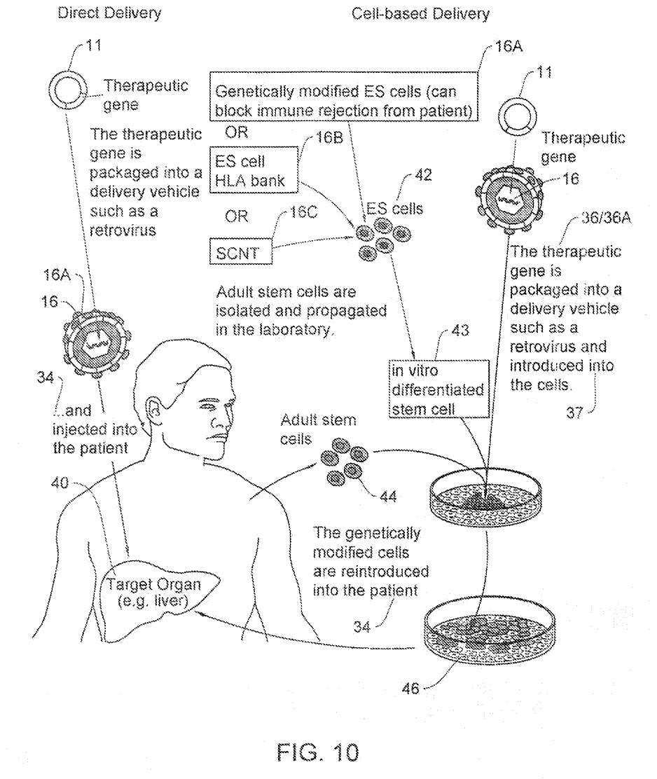

Specific genes for selected protein production may be cultured with a stem cell 11 of choice (see FIG. 10) which will then produce a supernatant composed of an exosome-gene complex 16A/39 (see FIG. 9). This exosome-gene complex may also be used as a therapeutic gene therapy on its own or in combination with the stem cell gene complex together for a specific cellular effect, organ or tissue regenerative effect or general systemic effects.

The effects of the central aging clock (see FIG. 11) located in the hypothalamus 48 and the memory center in the hippocampus 50, control multiple aspects of the body's natural rhythms related to health and aging. Loss of neural stem cells 44 overtime with the aging process causes these centers to dysregulate and eventually become inefficient to maintain normal health. Restoration of the cells lining the hypothalamus 48, the neural stem cells 44, with gene enhanced stem cells 34 like mesenchymal stem cells 38 (see FIG. 2) and there exosomes 39 (see FIGS. 2 and 3) loaded with the klotho gene 41 (see FIG. 4), can rejuvenate the function of the hypothalamus 48 to reset the central aging clock 49, as well as the hippocampus 50 to enhance memory and cognition 54 normally lost with the aging process. See FIG. 11 recent articles published in NATURE Journal by Jordan and Kai, have documented the importance of restoring stem cell function in select regions of the brain to enhance memory, cognitive function and the general aging process.

Described in this gene therapy technique to treat or prevent a disease or physiological function loss, caused by the human aging process. This is accomplished by inserting a specific gene into a patient's stem cell of choice. Stem cells can be genetically modified to carry therapeutic genes 34. These modified or enhanced stem cells can then be used to regenerate damaged tissues, organs or even restore cellular function (see FIGS. 9-10). The techniques described herein can be accomplished via an AAV (attenuated adenovirus) or CMV (cytomegalovirus) vector delivery systems 36A for long-term functional enhancement (see FIG. 9) or, a plasmid delivery system, for a short-term delivery system (see FIG. 10).

Both vector types can use selected genes of choice delivered into stem cells of different cell lineage. In this case we are describing human adult stem cells (ASC), both allogeneic and autologous stem cells including but not limited to hematopoietic stem cells (HSC), endothelial progenitor cells (EPC), and mesenchymal stem cells (MSN) 38 (see FIG. 2).

The source of these stem cells can be from peripheral blood, bone marrow or fat cells, from umbilical cord or other human tissue sources or induced pluripotent stem cells. This can be accomplished for human sources most readily via apheresis collection after mobilization with any number of mobilizing agents including neurogenic to obtain a combination of multiple stem cell types 43.

The goal of this process is to deliver or insert a gene of choice within the nuclear compartment of the stem cell, as an episome 39, virtually none integrating with the original nuclear DNA 18 (see FIGS. 2 and 9). This acts as an additional artificial mini-chromosome 14. These transfected cells can then be used systemically or locally to enhance stem cell function or suppress a given protein or restore specific cellular function as in increased muscle mass 52 (see FIG. 11) or enhanced cognition 54 (see FIG. 11) as well as immune enhancement 13.

The transfected cells 34 (see FIG. 9) can then be culture expanded to create more numbers of the enhanced cells type for specific purposes, including the exosome and protein components 40/55 and then re infused intravenously, sprayed intra nasally, and/or injected into aged local tissues, injected into lymphatics or the spinal canal 53 as well as other anatomical sites (see FIG. 11).

More specifically, certain stem cell lines can be treated with gene transfection with specific genes for a specific purpose. For example, hematopoietic stem cells, which form all cell types involved in immune system 13, can be treated with hTERT 34/39 to extend their telomere length 39 and allow them to make more copies of themselves for a much longer time than normally possible, therefore helping to restore immune function and avoid immunosenescence, which occurs to all human adults as they age. hTERT or telomerase 34/39 (see FIGS. 4 and 9) is a ribonucleoprotein polymerase that maintains telomere ends by addition of the telomere repeat TTAGGG 28, 26, 22 (see FIG. 4). The enzyme consists of a protein component with reverse transcriptase (hTERT) activity 16A/34, encoded by this gene, and an RNA component 46 that serves as a template for the telomere repeat (see FIG. 9).

Mesenchymal stem cells 38 (see also FIGS. 1 and 2) can be treated with follistatin protein 4 7 and/or PGC-1 alpha (peroxisome) 23 which can be injected either locally into muscle or given IV systemically to restore lost muscle mass and strength 52.

These same mesenchymal stem cells 38 may also be given to avoid sarcopenia and frailty that occur in all adults over time. The same mesenchymal stem cells can also be transfected with alpha klotho or other klotho subtypes 41 (see FIG. 4) and injected IV along with focused ultrasound which can help guide them to specific brain centers like the hypothalamus and hippocampus to restore the central aging clock 49 nucleus involved in multiple physical functions. (see FIG. 11) to help restore the neural nuclei responsible for maintaining general homeostasis of the aging process including memory decline as well as dementia and Parkinson disease.

Endothelial progenitor cells and other precursors to blood vessel formation can be transfected with hTERT 34/39 also to enhance blood vessel (transferring) 53A repair cells (see FIG. 11) and angiogenesis which is lost over time due to the aging process.

Selective immune cells like natural killer cells can also be transfected with hTERT 34/39 to function as a senolytic therapy to remove senescent cells, remove viral infections, as well as cancer cells and bacterial infections.

The concepts presented here are not limited to the genes mentioned in this application but may apply to multiple other genes that can be used for different organ regeneration like the liver 55, thymus, pancreas and other organs (see FIG. 11) and tissues not mentioned here. In general, the general process described here can be used to enhance stem cell function not mentioned in this preliminary application.

The general concepts revealed here can also be in multiple combinations including all genes with select stem cells to create a general gene stem cell therapy to prevent functional loss for each individual. These techniques can be applied not just to humans but to other non-human primates and animals in general including dogs, cats, and horses.

The intended use of this therapy is to create enhanced stem cell function on a long-term or permanent basis using different gene-based transfection technologies and different plasmid-based gene transfection technologies. Both technologies may incorporate a start or stop gene or other technologies to control the magnitude of gene expression desired for a given purpose or amount of time if needed for a given condition or effect.

The use of exosome products 39 from the transfected stem cell 36/43 can also be used for therapeutic effect with or without the transfected stem cells.

In Step 3, gene enhanced stem cell (GES) function is reprogrammed at the DNA level thereby restoring methylation and acetylation patterns, as above mentioned, more youthful function levels. This step is accomplished using a number of various techniques including AAV or CMV vectors or other gene transfer techniques.

Epigenetic reprogramming can be accomplished with repurposed medications as well as heterochronic cell culture techniques described by Giampapa in his previous patents. All such methods operate to reprogram a youthful state, specifically with genes that also provide proteins necessary to avoid or delay the normal declines in physical function all humans suffer from as they grow older.

At this point in the process specific genes can be chosen to selectively enhance a specific type of stem cell activity (see FIG. 8) to correct the physical condition lost with the aging process to a more normal level or even beyond the standard level of inherited gene activity to more rapidly restore the damage lost by the general aging process and the lost function a person is suffering from.

These losses of physical function that most frequently occur in. Humans are directly related to the loss of specific gene function and their related proteins along with the loss of stem cell numbers. This also causes future age-related disease processes and a decrease in health-span and longevity. They are not limited to the following conditions.

Sarcopenia and frailty: with the use of gene enhanced mesenchymal stem cells 38 with follistatin 47 (see FIG. 2). lmmunosenescece: with the use of hTERT enhanced hematopoietic stem cells 38.

Cognitive Decline: with the use of klotho cell 41. Enhanced mesenchymal stem cells with or without focused ultrasound therapy.

Loss of cellular and physical energy: with the use of PGC 1—alpha enhanced stem cells. The PGC1-alpha gene 23 is a protein coding gene involved in maintaining pluripotency by organizing genome-wide and associated with certain diseases.

Chronic inflammation: with a combination of all stem cell and somatic cell types with follistatin 47.

Loss of DNA repair function. With the use of a combination of all stem cell types and somatic cells with PARP and other DNA repair genes. PARPs are a family of related enzymes that share the ability to catalyze the transfer of ADP-ribose to target proteins. PARPs play an important role in various cellular processes, including modulation of chromatin structure, transcription, replication, recombination, and DNA repair.

Loss of liver function 55 with LDGT genes, and the use of retrotransposons of different varieties of PhiC31, integrate 59 and CASmRNA. Retrotransposons are evolutionary widespread genetic elements that replicate through reverse transcription of an RNA copy and integrate the product DNA into new sites in the host genome 18.

PARP provides a role as a first responder in the repair of DNA damage, including histone 21 (methyl), decompaction of chromatin structure 208 and 20C. See FIGS. 5-7.

Further, in FIG. 10 are provided genetically modified ES cells 42 are human leukocyte antigens (HLAs) 168 which are a group of genes that help the immune system distinguish between the body's own proteins and those from foreign invaders, such as viruses and bacteria. Alternatively, one may make use of somatic cell nuclear transfer (SCNT) 16C is the process of transplanting nuclei from adult cells into oocytes or blastocysts and allowing them to grow and differentiate, producing pluripotent cells. FIG. 10 illustrates SCNT 16C. This process has both reproductive and therapeutic implications.

In Step 4 programmed stem cells are set forth in the following potential areas:
i. rein-fusion using reprogrammed stem cells RSC;
ii. using directly injection of RSC; or
iii. using gene enhanced RSC,

whereas an IV peripheral line infused into the patient with the ASC for over a period of about 30 minutes; or whereas the gene enhanced cell RSC is directly injected into a specific issue type.

The patient is monitored with blood pressure 53A, pulse rate and oxygen levels throughout the reinfusion process monitoring treatment effectiveness and adjustment of the therapy described specific blood and physical testing can be accomplished over time to document the effectiveness of the treatment described here and a second treatment can be given to further improve the desired correction of the physical deficits being on the level of the new protein being produced. A method of delivery of the reprogrammed and gene enhanced stem cells are provided. These reprogrammed and gene enhanced stem cells are reinfused via system circulation using an IV process in the person to correct any number of age-related functional decline issues or age-related disease processes including the general aging process.

Although the present invention has been illustrated and described herein with reference to preferred embodiments and specific examples thereof, it will be readily apparent to those of ordinary skill in the art that other embodiments may perform similar functions and like results. All such equivalent embodiments and examples within the spirit and scope of the present invention are contemplated thereby and are intended to be covered by the following claims.

1-20. (canceled)

21. A method comprising:
(a) collecting and isolating mesenchymal stem cells (MSCs) from a donor or a patient,
wherein the MSCs are derived from bone marrow, adipose tissue, or peripheral blood following mobilization into the bloodstream;
(b) generating reprogrammed stem cells (RSCs) by exposing the MSCs to one or more histone-modifying agents that alter methylation and/or acetylation patterns;
(c) producing gene-enhanced stem cells (GESs) by transfecting the RSCs with a vector, wherein the vector comprises an adeno-associated virus (AAV), a cytomegalovirus (CMV), or a plasmid DNA vector,
wherein the vector includes a gene which encodes human telomerase reverse transcriptase (hTERT) and/or telomerase RNA component (TERC);
(d) culturing the GESs to express the human telomerase reverse transcriptase (hTERT) and/or telomerase RNA component (TERC);
(e) isolating exosomes from the GESs; and
(f) administering the isolated exosomes to a patient.

22. The method of claim 21, wherein the step of generating RSCs comprises promoting histone demethylation by delivering JMJD3 to the MSCs.

23. The method of claim 21, wherein the step of generating RSCs comprises promoting histone acetylation by treating the MSCs with a histone deacetylase (HDAC) inhibitor.

24. The method of claim 21, wherein the step of generating RSCs comprises promoting histone demethylation by delivering JMJD3 to the MSCs and promoting acetylation by treating the MSCs with an HDAC inhibitor.

25. The method of claim 21, wherein producing the GESs further comprises transfecting the RSCs to express a second gene.

26. The method of claim 25, wherein the second gene is the FST gene, which encodes follistatin.

27. The method of claim 25, wherein the second gene is the KL gene, which encodes the klotho proteins.

28. The method of claim 25, wherein the second gene is the PPARGC1A gene, which encodes PGC-1α.

29. The method of claim 21, further comprising storing the isolated MSCs in a sterile cryopreservation container maintained at a temperature between −100° C. to −60° C.

30. The method of claim 21, wherein the step of collecting MSCs further comprises apheresis.

31. The method of claim 21, further comprising measuring a degree of telomere elongation in the GESs following the step of culturing the GESs and before the step of isolating the exosomes.

32. The method of claim 21, wherein the step of administering the isolated exosomes comprises intravenous infusion, direct tissue injection, or intranasal delivery.
